(19) United States
(12) Patent Application Publication
(10) Pub. No.: US 2024/0303806 A1
(43) Pub. Date: Sep. 12, 2024

(54) SYSTEM AND METHOD FOR DENTAL IMAGE ACQUISITION AND RECOGNITION OF EARLY ENAMEL EROSIONS OF THE TEETH

(71) Applicant: GLAXOSMITHKLINE CONSUMER HEALTHCARE HOLDINGS (US) LLC, Wilmington, DE (US)

(72) Inventors: Ann Theodore BALLESTEROS, Warren, NJ (US); Dilshan Maduranga GANEPOLA, Skillman, NJ (US); Roshan Hashantha HEWAPATHIRANA, Skillman, NJ (US); Achala Upendra JAYATILLEKE, Skillman, NJ (US); Stephen William PITT, Warren, NJ (US); Buddhika Tharindu RANASINGHE, Skillman, NJ (US); Pandula Anilpriya SIRIBADDANA, Skillman, NJ (US); Shanil Anthony SINGARAYAR, Skillman, NJ (US); Tithila Kalum WETTHASINGHE, Skillman, NJ (US)

(21) Appl. No.: 18/258,747

(22) PCT Filed: Dec. 21, 2021
(86) PCT No.: PCT/US2021/064589

§ 371 Date: Jun. 21, 2023

Related U.S. Application Data
(60) Provisional application No. 63/128,926, filed on Dec. 22, 2020.

(51) Int. Cl. G06T 7/00; G06T 1/00

(52) U.S. Cl. CPC G06T 7/0012; G06T 1/0007; G06T 2207/20081; G06T 2207/20084; G06T 2207/30036

(57) ABSTRACT

The system and method of the present invention processes a raw image of a person's teeth captured through a camera according to given specifications by using a uniquely trained convolutional neural network (CNN). The system and method identify early erosions and their location on the raw image of the teeth.

### BACKGROUND OF THE INVENTION

### 1. Field of the Invention

The present invention is directed to a recognition and classifying system and method for early enamel erosions of the teeth. The present invention is particularly directed to such a recognition and classifying system and method based on image analysis to train and use a convolutional neural network (CNN).

### 2. Description of Related Art

Convolutional neural networks for image classification are described by, e.g., P. Pinheiro, and R. Collobert, “Recurrent convolutional neural networks for scene labeling. Proceedings of the 31 st International Conference on Machine Learning, Beijing, China, 2014. JMLR: W&CP volume 32 (pp. 82-90); and I-Saffar et al., “Review of Deep Convolution Neural Network in Image Classification,” 2017 International Conference on Radar, Antenna, Microwave, Electronics, and Telecommunications, University Malaysia Pahang Institutional Repository. For a discussion of deep regression techniques, see Lathuiliere et al., “A Comprehensive Analysis of Deep Regression,” at arXiv:1803.08450v3 [cs.CV] 24 Sep. 2020.

### SUMMARY OF THE INVENTION

Oral diseases affect upwards of 3.58 billion people worldwide. Among the most common are abrasions and erosions, carries of permanent teeth, calculi, gingivitis, plaque, and stains. Early diagnosis of these dental conditions is important.

Dental erosion is defined as a chemical process that involves the dissolution of dental hard tissue, such as enamel and dentine, by acid not derived from bacteria. The dissolution occurs when the surrounding aqueous phase is undersaturated with tooth mineral. Although the World Health Organization (WHO) lists dental erosion in the International Classification of Diseases, clinicians tend to disregard erosive tissue loss as a disease per se. One reason is because erosion and physical wear also contribute to the physiological loss of dental hard tissue throughout a person's lifetime.

Abrasion is the progressive loss of hard tooth substances caused by mechanical actions other than mastication or tooth-to tooth contacts.

Significantly, the dissolution or loss of dental hard tissue is irreversible. Moreover, the dissolution or loss of dental hard tissue can lead to lesions and serious dental problems if allowed to progress.

Early dental hard tissue erosion does not cause clinical discoloration or softening of the tooth surface. As such, early dental hard tissue erosion is difficult to detect either visually or by tactile sensing. In addition, early hard tissue dental erosion may not manifest any symptoms, or the symptoms may be minimal and thus difficult to assess.

However, dental morphology changes given time and continuous exposure to acidic chemicals, including those acids contained in soft drinks. Eventually, a lesion will form, and the erosion will manifest as a matte appearance. Color will also deteriorate, and vary from yellow to brown, as the lesion erodes or approaches the dentin. Also, at this stage, teeth are more sensitive to heat changes. The erosive lesion may also become rough and form small concavities.

Early diagnosis of dental erosion is important. Dental erosion can be prevented either with proper dental cleaning or by avoiding acidic foods that give rise to such erosions.

The assessment of erosive wear is difficult since surface loss generally progresses slowly and requires extended periods of observation to detect changes. Another challenge is the identification of a stable reference from which loss of tooth substance can be gauged.

Basic erosion wear examination (BEWE) and visual erosion dental examination (VEDE) have been developed to ensure coordination between clinicians in grading erosion. Various clinical indices have been designed to detect and quantify tooth surface loss due to erosion from the other causes. Most indices were designed with the clinical diagnosis and recording and monitoring of erosive wear lesions as the focus. These indices rely on subjective clinical descriptions and may not be as accurate as desired when the morphological changes are minimal.

Currently, clinical appearance is the most important diagnostic feature. As discussed above, dental professionals may not readily recognize very early stages of dental erosion, and may dismiss minor tooth surface loss (TSL) as a normal and inevitable occurrence of daily living and, thus, incorrectly determine that no specific intervention is needed. Only at the later stages of the disease in which dental hard tissue erosion becomes evident by a routine examination, namely when dentine is exposed, and the appearance and shape of the teeth are significantly altered, does treatment commence.

Given that dental erosions are mainly identified using visual appearance, consumers must rely on the expertise of their dental practitioners to determine whether there is dental erosion.

In addition to their expertise, the dental practitioners upon which consumers rely have access to sophisticated dental image capturing systems. These systems, which are generally costly, are intended to be used by professionals in professional settings. These systems often include several components in addition to the image capture device itself.

An example includes sophisticated illumination devices.

The output of these systems is intended for professionals and requires specialized training. These systems can be bulky requiring space and specific environmental adjustments. These systems are costly and cost prohibitive for a consumer. These systems would require a consumer to invest a considerable amount of money for merely an early stage detection of enamel erosion. Moreover, these systems require technical maintenance beyond the capability of a typical consumer.

The present invention provides a system and method for the detection of dental erosion.

The present invention also provides a system and method that is an intelligent tool that can identify early enamel erosion.

The present invention further provides such a system and method for routine dental practice to enable the determination of the progression of dental erosion.

The present invention provides a system and method that uses a convolutional neural network (CNN). CNNs are deep learning algorithms that can train large datasets with millions of parameters. Deep learning algorithms are designed in such a way that they mimic the function of the human cerebral cortex. These algorithms are representations of deep neural networks, i.e. neural networks with many hidden layers.

The present invention provides such a system and method that aims to model high-level abstractions in data by using a deep graph with multiple processing layers for the automatic extraction of features. Such an algorithm automatically grasps the relevant features required for the solution of the problem, which, in turn, reduces the work of the dental specialist.

The present invention provides a system including an image capture device, a display device, and processor.

The present invention also provides a Neural Network Algorithm that takes metadata as an input and processes the metadata through several layers of a non-linear transformation to compute an output classification.

The present invention provides an effective, convenient and cost-effective method of acquiring dental images in the privacy of the consumer's own personal space or home.

The present invention provides a hands-free smart oral image acquisition and display system capable of incorporating visible and near infrared image capturing mechanisms and illumination sources.

The present invention provides an image acquisition system that can be used by consumers without prior training in their own home environment.

The system can integrate with a consumer's own smart phone or a smart display device to show a captured image in real time and transmit the same to a cloud-based processing system before re-directing the processed image back to the consumer's display.

The system makes it possible for the consumer to capture images of the teeth, usable for processing and identifying dental pathologies by a cloud-based image processing system, in the consumer's own private space without having to be trained or having to operate the device manually.

The present invention further provides such a system and method that imparts self-care approaches among consumers. Such self-care approaches can include lifestyle adjustments that halt the progression of enamel erosion, and even other oral diseases.

Advantageously, the system and method benefits the consumer in that the consumer experiences fewer oral symptoms throughout life, but also the consumer spends less time and money to manage oral health.

The above is not intended to describe each disclosed implementation, as features in this disclosure can be incorporated into additional features as detailed herein below unless clearly stated to the contrary.

### DESCRIPTION OF THE DRAWINGS

The accompanying drawings illustrate aspects of the present invention, and together with the general description herein, explain the principles of the present invention. As shown throughout the drawings, like reference numerals designate like or corresponding parts.

FIG. 1 is a system according to the present invention.

FIG. 2 shows a CNN diagram according to the present invention.

FIG. 3 shows a convolution, performed by 3×3 kernel on original image to produce a 6×6 matrix.

FIG. 4 is illustrative of types of pooling of the CNN according to the present invention.

FIG. 5 depicts a process according to the present invention for identifying early erosion and its location on a raw image of teeth.

FIG. 6 is a perspective view showing an image capturing device according to an embodiment of the present invention.

FIG. 7 shows an exemplary display device according to the present invention.

FIG. 8 shows another exemplary display device according to the present invention.

FIG. 9 shows an operating environment of the system and method of the present invention.

FIG. 10 is a flow chart of a method for capturing dental images according to the present invention.

### DETAILED DESCRIPTION OF THE INVENTION

The system and method of the present invention processes an image of a person's teeth captured through a camera by using a uniquely trained convolutional neural network or CNN. Thus, the system and method can identify early erosions, as well as the erosion locations on the teeth.

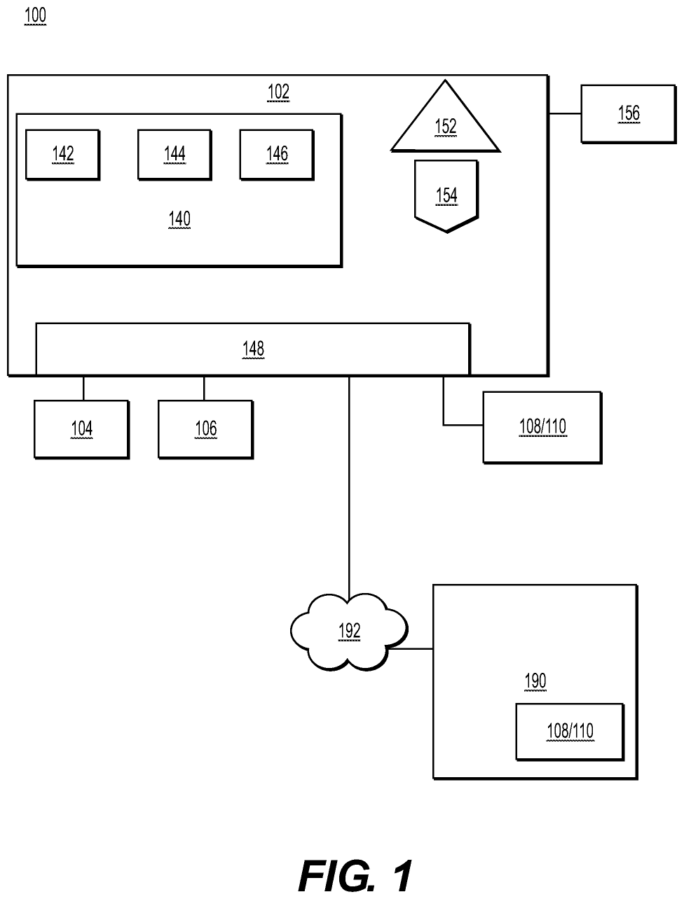

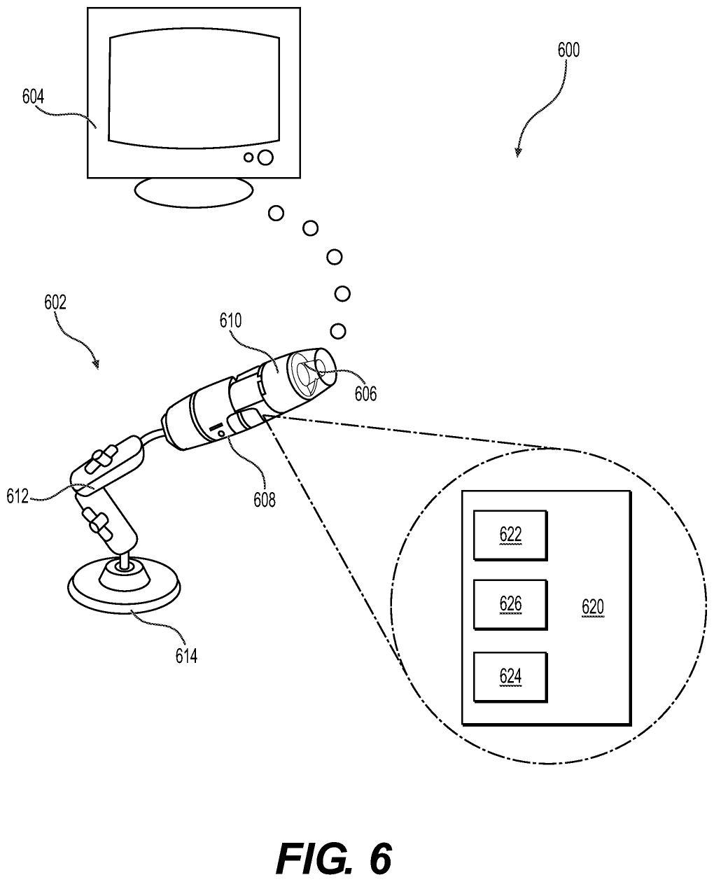

Referring to the drawings, and in particular to FIGS. 1 and 6, there is shown a system for training a CNN according to the present invention and generally referenced by the numeral 100, and a system identifying early erosions generally referenced by the numeral 600, respectively.

Referring to FIG. 1, system 100 includes the following exemplary components that are electrically and/or communicatively connected: a computing unit 102, a digital image capture device 104, an image processor 106, and a trained neural network model 108.

Trained neural network model 108 can be local or on a server 190 that is in communication over a network 192 such as the internet.

Computing unit 102 can include: a control unit 140, which can be configured to include a controller 142, a processing unit 144 and/or a non-transitory memory 146. Computing unit 102 can also include an interface unit 148, which can be configured as an interface for external power connection and/or external data connection, a transceiver unit 152 for wireless communication, antenna(s) 154, and a display 156. The components of computing unit 102 can be implemented in a distributed manner.

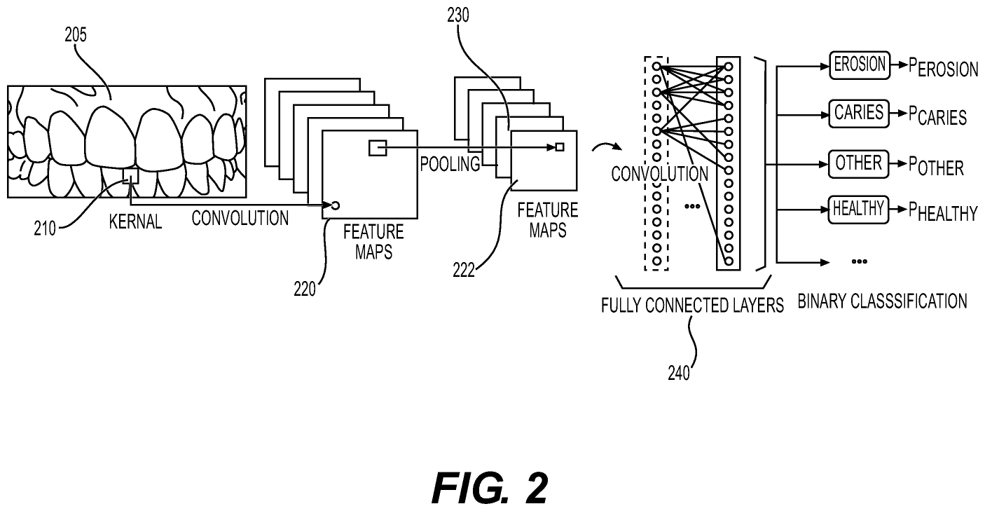

Referring to FIGS. 1 and 2, image processor 106 of system 100 is operatively connected or coupled to a network 192. Image processor 106 is configured to receive an image that can include one or more dental images 205 of a person from a digital image capture device 104. Image processor 106 is further configured to provide the images 205 to train or to a trained neural network model 108, such as CNN 110 of FIG. 1.

CNN 110 is a deep learning algorithm. CNN 110 can receive as input an image. CNN 110 can also classify the image 205 or identify and differentiate objects in the image 205.

CNN 110 is configured to learn to and to determine enamel erosion of each tooth based on the image received from the image processor. CNN 110 or the neural network model 108 also learns to and determines the amount of grading associated with enamel erosion so that system 100 can provide feedback.

In one embodiment shown in FIG. 2, CNN 110 has four main components. The four components are an input layer 210, a convolutional layer 220, a pooling layer 230, and a fully connected layer 240. Each layer 210, 220, 230 and 240 has neurons.

Input layer 210 contains the pixel value of the image 205.

Convolutional layer 220 holds the main features that are extracted by the process of convolution. The main task of convolution is to reduce the image size and extract main features. Convolution is achieved using a Kernel/Filter that is N×N matrix. Output of the convolution is a feature map 222.

System 100 extracts prominent features with pooling layer 230.

Fully connected layer 240 contains neurons that are directly connected to the neurons in adjacent layers. Fully connected layer 240 performs a classification task, to make a prediction.

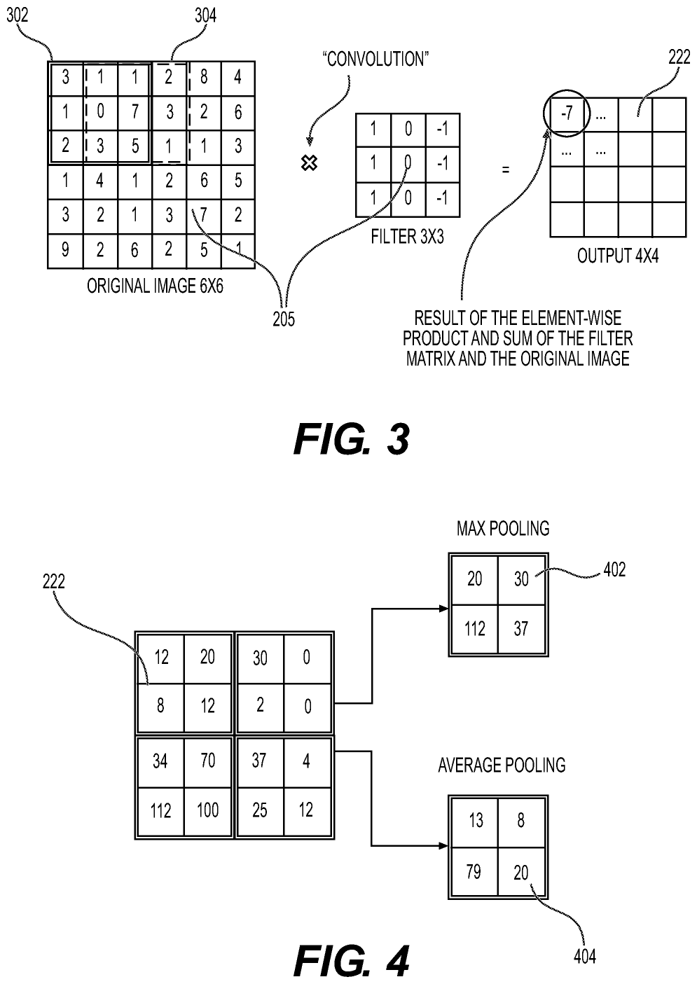

FIG. 3 illustrates the convolution calculation process, and how it is performed by 3×3 kernel on an original image to produce a 6×6 matrix (see, e.g., A. D. Nishad, “Convolution Neural Network (Very Basic)/Data Science and Machine Learning/Kaggle, at https://www.kaggle.com/general/171197). FIG. 4 is an illustrative example of a type of pooling of the CNN and how the maximum and average values are calculated. In FIG. 3 and FIG. 4, the numbers in the grids illustrate how an image may be represented in the neural network as it is processed.

Convolution is shown in FIG. 3 as square 302 (3×3 matrix) that shifts to the right on the input image, from a left side to a right side, resulting in a square 304. Once convolution reaches the rightmost edge of the image 205, a shift down by one row occurs and the process to scan the entire image repeats. At every shift to the right side, a matrix multiplication is performed between the kernel and the image over which the kernel is hovering. The sum of the results is inserted to a new grid, feature map 222.

System 100 extracts prominent features by pooling.

As shown in FIG. 4, pooling further reduces the size of feature map 222, to advantageously reduce the computational power required for processing. Common types of pooling are max pooling 402 and average pooling 404. Max pooling 402 returns the maximum value in the region covered by the kernel. Average pooling 404 returns the average value of all values in the region covered by the kernel.

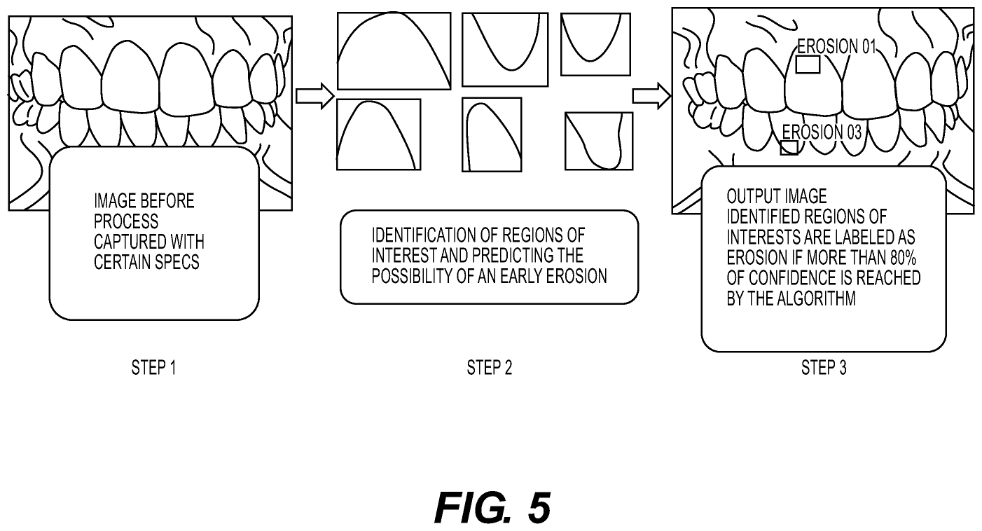

The process according to the present invention will now be described with reference to FIG. 5.

In Step 1, a raw image is captured using a camera, and is submitted to a trained CNN.

In the examples, the raw image clearly shows two rows of teeth from a prospective front on view with appropriate lighting that does not distort the appearance of the teeth by a shadow, discoloration, over or under exposure.

In the examples, the area of the image taken up by the teeth is at least 60%.

In the examples, the image has an aspect ratio of 4:3 and a minimum resolution of 800×600 pixels.

In Step 2, the CNN processes the submitted image to identify one or more areas where early enamel erosions are likely to be present.

The CNN is trained by images tagged by a trained clinician using his/her expertise and judgment. To train CNN 110, two sets of dental images were obtained and tagged.

The images were obtained from about 700 patients.

Tagging can be carried out according to the BEWE scoring system, as described by Bartlett et al., “Basic Erosive Wear Examination (BEWE): a new scoring system for scientific and clinical needs,” Clin Oral Invest (2008) 12 (Suppl 1):S65-S68, incorporated by reference. The four level score grades the appearance or severity of wear on the teeth from no surface loss (0), initial loss of enamel surface texture (1), distinct defect, hard tissue loss (dentine) less than 50% of the surface area (2) or hard tissue loss more than 50% of the surface area (3).

Tagging of dental erosive wear can also be carried out according to the Visual Erosion Dental Examination (VEDE) system, having the with the following criteria: grade 0=no erosion; grade 1=initial loss of enamel, no dentine exposed; grade 2=pronounced loss of enamel, no dentine exposed; grade 3=dentine exposed, <⅓ of the surface involved; grade 4=dentine exposed, ⅓-⅔ of the surface involved; grade 5=dentine exposed, >⅔ of the surface involved, see, e.g., Mulic et al., “Reliability of two clinical scoring systems for dental erosive wear,” Caries Res 2010; 44(3):294-9, incorporated by reference.

In the present example, a dentist tagged the images using his clinical judgment consistent with BEWE or VEDE scoring, regarding the condition observed in the images.

In the first set of dental images, the training focus was based on observations of early erosions. In the first set of dental images, the training focus was also on erosions but further included abrasions.

The tagging therefore indicated an area or position, an extent or magnitude, and a color change within the area and any surface changes. Stated another way, the tagging was based on clinical evidence.

Conditions trained for include early enamel erosion, gingivitis, abrasions and erosions, carries of permanent teeth, calculi, plaque, and stains.

About 1000 to 1500 data points were used to train for each condition. Gingivitis training used about 700 data points given its rarity.

By way of nonlimiting example, the tagging mechanism can indicate the risk, sensitivity, classification and degree.

In the examples, the algorithm on which CNN 110 is based can re-size the images fitting the optimum processing capability of the CNN. The algorithm can use a pre-defined set of anchors specific to recognizing early enamel erosions at ratios 1:1, 1:1.4 and 1.4:1 in the scales of 24, 46 and 64 during region proposal.

In such examples, the algorithm can have a unique overlap threshold and an algorithm confidence threshold that fits the need of identifying early enamel erosion.

In Step 3, CNN 110 generates a processed image with tags as indicated in Step 2. The generation of the processed image can be in real time. The processed images, including tags, are transmitted from server 190, where the CNN 110 is based, to a display 156 shown in FIG. 1.

In the examples, the process described in Steps 1 to 3 is managed by a software application made for digital devices, such as smart devices (e.g. ANDROID® or IOS® based) having their own digital image capture device (camera). The software application facilitates capturing of the image, storage of the image, transmitting the image to server 190 where CNN 110 is located, receiving the processed image from the server 190 via network 192, and displaying the processed image for the consumer on a display 156.

Referring to FIG. 6, system 600 will now be described.

System 600 includes an image capture device 602 for capturing oral images and a display device 604 that provides a user with the device functions of previewing, photographing, storing, analyzing and forwarding oral images. Image capture device 602 includes a camera 606 that is sensitive to visible light and/or near-infrared light and a light source 608 disposed in or about a housing 610.

Image capture device 602 is configured to capture an image of the front and inside surfaces of the teeth as a user poses with open mouth, teeth visible and positioned at a pre-defined distance. The process of using image capture device 602 is guided by the display device 604 that can include visible guidelines and instructions and provides functionality for automated and handsfree operation.

Image capture device 602 is communicatively connected to display device 604. In the examples, image capture device 602 is communicatively connected to display device 604 by wireless communication. In other examples, image capture device 602 is communicatively connected to display device 604 by wired communication.

In the exemplified embodiments, image capture device 602 captures images of the teeth automatically and transmits to display device 604 in real time. This enables a user to look at display device 604 and adjust the positioning. Image capture can be triggered by voice, for example a user pronouncing “eee” or “aahh” for few seconds while teeth are visible on display device 604. This functionality, “teeth detection function”, can be software or hardware.

Light source 608 can be a white LED and/or a near infrared LED. Light source 608 can be switched on and off and provides two different wavelengths of illumination for the system, allowing the acquisition of visible and/or near-infrared images.

Light source 608 is controlled by an application of display device 604. The data processing unit is further connected to the Bluetooth module, and through the Bluetooth module, the data processing unit 624 can connect with display device 604 to transmit data.

Preferably, the LEDs are of visible and/or near infrared wavelengths of 940 nm, 1000 nm and 1300 nm. Illumination is synchronized with the capturing of the image and is controllable by display device 604.

Further, within housing 610, there is a main control circuit board 620 that includes a battery 622, a data processing unit 624, and a Bluetooth module 626. Data processing unit 624 is communicatively connected to camera 606 and controls the camera to capture an image. Data processing unit 624 is also connected to light source 608.

Housing 610 can be optionally attached to an adjustable stand 612 that is supported on a platform 614. By mounting the devices on fixed and adjustable supports, the system 600 avoids the need for the user to handle the image capturing device 602. This allows the user to pose freely without having to coordinate mechanical or manual operation facilitating the user to position his/her mouth relative to the camera. Thus system 600 facilitates obtaining a high-quality dental image, for processing.

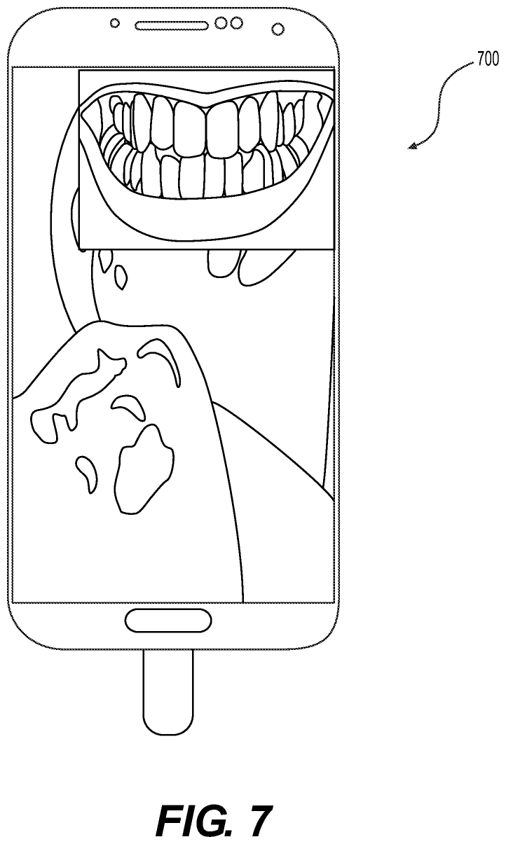

Display device 604 can be smartphone 700 as shown in FIG. 7.

Smart phone 700 is configured with logic and circuitry configured to perform one or more (and preferably all) of the functions of: guiding the user in taking an optimal image of the teeth; receiving the captured image from the image capturing device via Bluetooth; displaying the said image; providing guidelines and instructions to adjust the positioning of the teeth by the user; storing the image; transmitting the stored image to an image processor 106 or equivalent cloud based image processing system via the internet; receiving the processed images from the cloud based image processing system via internet; and displaying the processed image with tags identifying dental pathologies. It is advantageous for the smart phone to be a large-screen touch sensitive mobile phone for easy visibility.

Smart phone 700 can have a software application that is configured to facilitating the aforementioned functions, and preferably further including one or more (and preferably all) of the functions of: identifying visible teeth surfaces/outline, proper distance and ratio of the visible teeth; transmitting the image to and from the display device real time; and storing of the images. The software is preferably configured to provide a user with the device functions of previewing, photographing, storing, analyzing and forwarding oral images.

As discussed above, smart phone 700 is further configured in software to guide the user using voice, graphics, or both, in obtaining an optimal image by the teeth detection function.

The teeth detection function is a feature of the software that determines an appropriate ratio, distance and clarity of the image to be acquired prior to acquiring. The teeth detection function triggers the image capturing device either by identifying the open mouth and visible teeth or when the user creates a specific sound such as ‘eee’ while showing the teeth for a preset period of time such as 2, 3 or more seconds. The teeth detection function enables acquiring an image of the teeth of the user without the user having to manually operate.

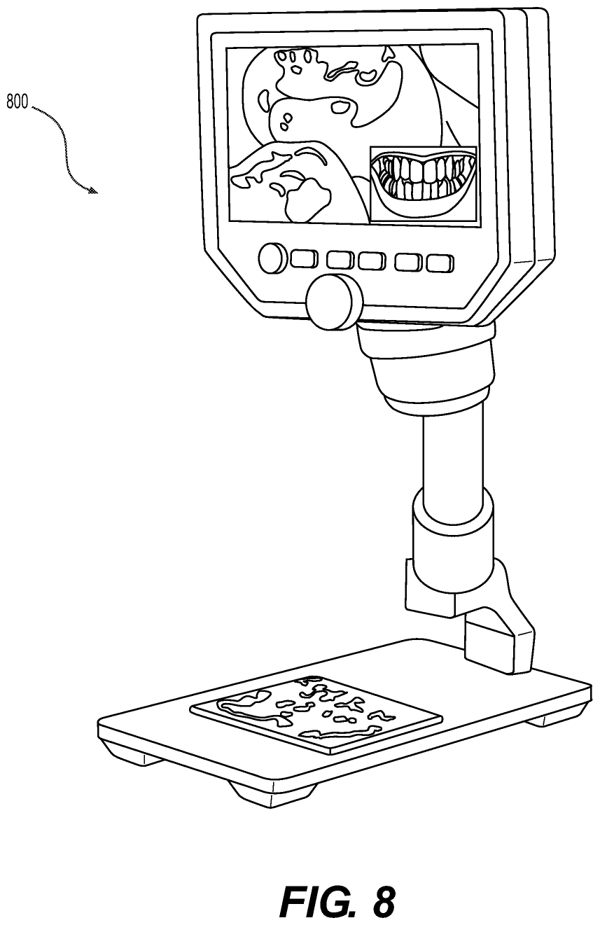

Alternatively, as shown in FIG. 8, display device 604 can be a special purpose display device 800 configured with logic and circuitry configured to perform one or more (and preferably all of) the functions of: guiding the user in taking an optimal image of the teeth; receiving the captured image from the image capturing device via Bluetooth; displaying the said image; providing guidelines and instructions to adjust the positioning of the teeth by the user; storing the image; transmitting the stored image to image processor 106 or an equivalent cloud based image processing system via the internet; receiving the processed images from image processor 106 or the cloud based image processing system via internet; and displaying the processed image with tags identifying dental pathologies.

Special purpose display device 800 can have a software application that is configured to facilitating the aforementioned functions, and further including being able to: identify visible teeth surfaces/outline, proper distance and ratio of the visible teeth; transmit the image to and from the display device real time; and manage storing of the images. The software is preferably configured to provide a user with the device functions of previewing, photographing, storing, analyzing and forwarding oral images.

The special purpose display device can further include a “teeth detection function” as smart phone 700.

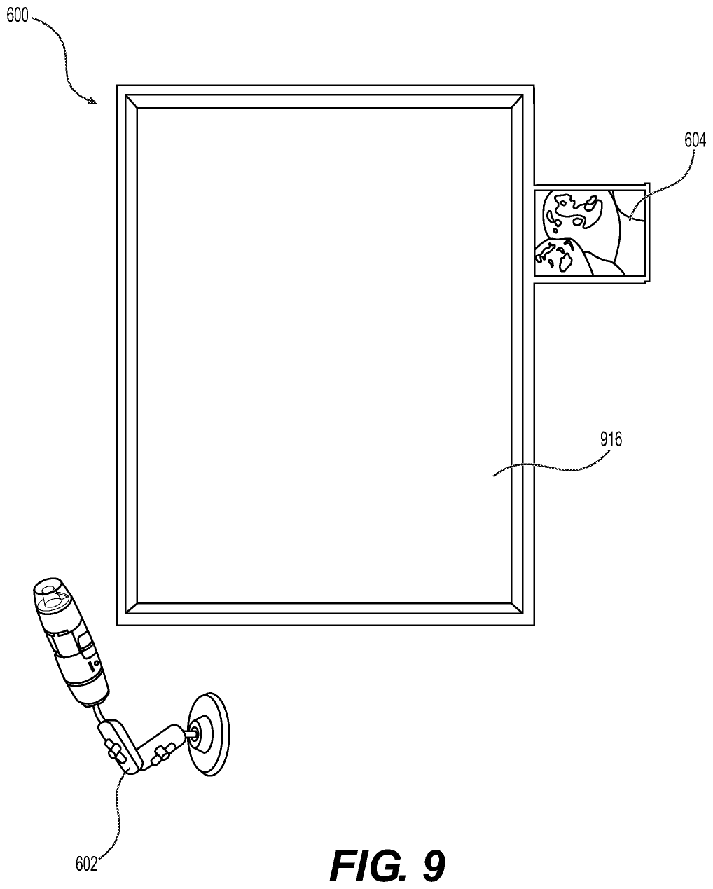

As shown in FIG. 9, image capture device 602 and display device 604, such as smart phone 700 or special purpose display device 800, can be connected or attached to a mirror such as bathroom mirror 916. A user can thus can readily incorporate system 600 into their daily dental routine in the comfort of their own bathroom.

System 600 is in communication with a network 192 (FIG. 1) such as the internet. The captured image can be transmitted through the internet by display device 604 to a cloud based image processing system or image processor 106, which can transmit back the processed image via the internet to the display device 604. The display device 604 can connect to the internet via an integrated wireless connection module.

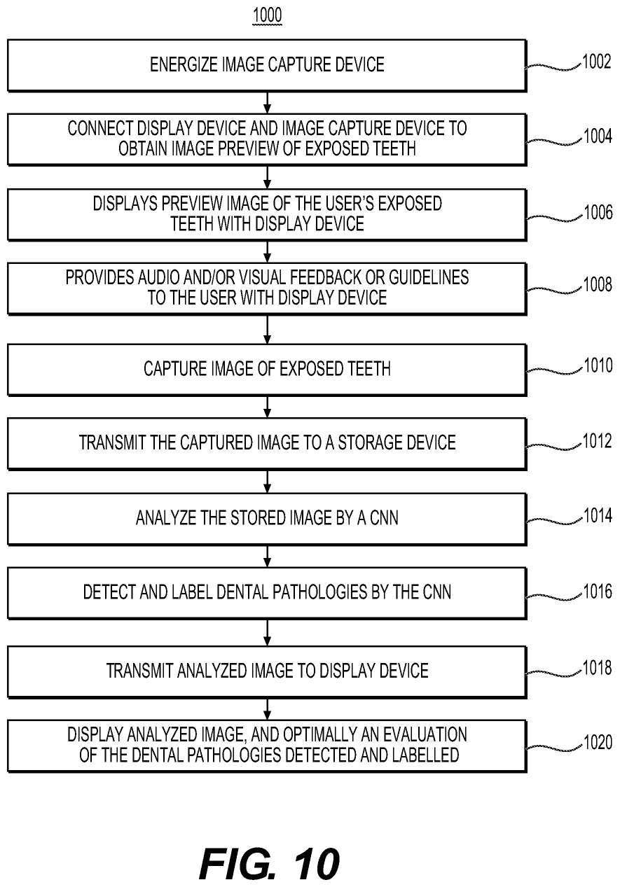

Operation 1000 of system 600 will now be described with reference to FIG. 10.

In step 1002, a user turns on image capture device 602 and thereby the camera and the illumination source. Thus, in step 1002, image capture device 602 is energized.

In step 1004, the user connects the display device 604 with image capture device 602 and positions the device. Image capture device 602 and display device 604 can also connect automatically based on a detected proximity therebetween. Thus, in step 1004, display device 604 communicatively connects to image capture device 602.

In step 1006, the user exposes the front/inside teeth to camera 606. This enables the user to preview the image in real time on display device 604. Thus, in step 1006, display device 604 displays a preview image of the user's exposed teeth captured by camera 606.

In step 1008, if necessary, the user adjusts the exposed teeth in a way that the teeth appear within the guidelines shown on display device 604 or according to voice instructions from the same display device. Thus, display device 604 provides audio and/or visual feedback or guidelines to the user.

In step 1010, the user holds the exposed teeth in position for a preset period of time to capture an image of the teeth or produces a sound such as ‘eee’ for a few seconds while the teeth are visible to activate the camera. Thus, in step 1010, an image of the exposed teeth is captured.

In step 1012, the display device/smart phone stores and transmits the captured image to an image processor storage device and/or a cloud based image processing system via the internet.

In step 1014, a trained convolution neural network (CNN) analyzes the image, for example CNN 110 of FIG. 1.

In step 1016, the CNN detects and labels dental pathologies.

In step 1018, the analyzed image is transmitted back to display device 604 via the internet.

In step 1020, display device 604 displays and stores the analyzed image together with an evaluation of the dental pathologies detected and labelled by the CNN during processing

The user can turn off image capture device 602 to end the session.

Based on the processed image and classification, the system 600 can provide the consumer with specific instructions as to the next course of action. Nonlimiting examples include cleaning and flossing instructions, special treatment for teeth recommendations, lifestyle adjustment advice and dental checkup reminders.

In particular, the invention in its various embodiments is described in the following numbered paragraphs:
(1) A system for training an image recognition algorithm for early enamel erosion detection, the system comprising: an image processor connected to a network, the image processor configured to: receive from a digital device, a set of images; tag one or more areas on each image of the set where there exists an indication of early enamel erosion; provide the tagged image to a neural network model to train a neural network model to recognize enamel erosion based on the tagged dental image; and detect enamel erosion from the trained neural network model.
(2) The system of Par. (1), wherein the trained neural network model is a regression deep learning convolutional neural network model.
(3) The system of Par. (2), wherein the regression deep learning convolutional neural network model is trained by dental images of persons associated with corresponding early enamel erosion images.
(4) The system of Par. (2), wherein the convolutional neural network: receives input data to be the object of recognition, performs object recognition, and outputs the object recognition result.
(5) The system of Par. (2), wherein convolutional neural network receives input data for object recognition, and wherein the object recognition is performed and outputs the process target recognition result.
(6) The system of Par. (5), wherein the target recognition process includes each convolution in a convolution layer, each neuron based on each input channel signal, data on each channel separately convoluted signal, a channel selection section signal, and a signal of the selected channel result of a convolution mapping feature to obtain characteristic information.

Thus, following multiple convolutions, a feature map is generated; then the regions of interest are extracted and fed into a fully connected layer; and finally classification is done and bounding boxes are created.
(7) The system of Par. (6), wherein the characteristic information obtained as a result of the output of the neuron is an input and an output of a convolutional next layer neuron.
(8) The system of Par. (1), further comprising a server and a network, wherein the trained neural network model is stored on the server.
(9) The system of Par. (1), further comprising a digital device, wherein the digital device is configured to capture the image including dental images of the person, and wherein the digital device is electrically coupled to the network.
(10) The system of Par. (1), wherein the image processor is further configured to evaluate the image determine a degree of enamel erosion of the person.
(11) The system of Par. (1), further comprising an electronic device to receive the detected enamel erosion of the person and receiving the input from the electronic device to a smart phone.
(12) An image acquisition system for early enamel erosion detection, the system comprising: an image capturing device; and a display device operatively connected to the image capturing device;
   wherein the image acquisition system is configured to: capture an image of a user's exposed teeth; transmit the obtained image to a trained CNN that analyzes the obtained image by detecting and labeling dental pathologies with the trained CNN to and yields an analyzed image; and receive and display the analyzed image on the display device.
(13) The system of Par. (12), further comprising a light source.
(14) The system of Par. (13), wherein the light source is configured to emit visible and near infrared light.
(15) The system of Par. (12), wherein the image capturing device is sensitive to visible and near infrared light sources.
(16) The system of Par. (12), wherein the image capture is based on a timer.
(17) The system of Par. (12), wherein the image capture is based on a voice command.
(18) The system of Par (1), wherein the enamel erosion detection uses a pre-defined set of anchors specific to recognizing early enamel erosions at ratios 1:1, 1:1.4 and 1.4:1 in the scales of 24, 46 and 64 during region proposal.
(19) A method for training an image recognition algorithm for early enamel erosion detection using the system of Par. (1).

It should be noted that the terms “first”, “second” and the like can be used herein to modify various elements. These modifiers do not imply a spatial, sequential or hierarchical order to the modified elements unless specifically stated.

As used herein, the terms “a” and “an” mean “one or more” unless specifically indicated otherwise.

As used herein, the term “substantially” means the complete or nearly complete extent or degree of an action, characteristic, property, state, structure, item, or result. For example, an object that is “substantially” enclosed means that the object is either completely enclosed or nearly completely enclosed. The exact allowable degree of deviation from absolute completeness can in some cases depend on the specific context. However, generally, the nearness of completion will be to have the same overall result as if absolute and total completion were obtained.

As used herein, the term “comprising” means “including, but not limited to; the term “consisting essentially of” means that the method, structure, or composition includes steps or components specifically recited and may also include those that do not materially affect the basic novel features or characteristics of the method, structure, or composition; and the term “consisting of” means that the method, structure, or composition includes only those steps or components specifically recited.

As used herein, the term “about” is used to provide flexibility to a numerical range endpoint by providing that a given value can be “a little above” or “a little below” the endpoint. Further, where a numerical range is provided, the range is intended to include any and all numbers within the numerical range, including the end points of the range.

While the present invention has been described with reference to one or more exemplary embodiments, it will be understood by those skilled in the art, that various changes can be made, and equivalents can be substituted for elements thereof without departing from the scope of the present invention. In addition, many modifications can be made to adapt a particular situation or material to the teachings of the present invention without departing from the scope thereof. Therefore, it is intended that the present invention will not be limited to the particular embodiments disclosed herein, but that the invention will include all aspects falling within the scope of a fair reading thereof.

What is claimed is:

1. A system for training an image recognition algorithm for early enamel erosion detection, the system comprising:
an image processor connected to a network, the image processor configured to:
receive from a digital device, a set of images;
tag one or more areas on each image of the set where there exists an indication of early enamel erosion;
provide the tagged image to a neural network model to train the neural network model to recognize enamel erosion based on the tagged dental image; and
detect enamel erosion from the trained neural network model.

2. The system of claim 1, wherein the trained neural network model is a deep learning convolutional neural network model, wherein an object of recognition is enamel erosion.

3. The system of claim 2, wherein the deep learning convolutional neural network model is trained by dental images of persons associated with corresponding early enamel erosion images.

4. The system of claim 2, wherein the deep learning convolutional neural network model is capable of receiving input data for the object of recognition, performing object recognition, and outputting the object recognition result.

5. The system of claim 1, further comprising a server and a network, wherein the trained neural network model is stored on the server.

6. The system of claim 1, further comprising a digital device, wherein the digital device is configured to capture the images, and wherein the digital device is electronically coupled to the network.

7. The system of claim 1, wherein the image processor is further configured to evaluate the images to determine the degree of enamel erosion.

8. The system of claim 1, further comprising an electronic device to receive the detected enamel erosion and transmit the input from the electronic device to a smart phone.

9. An image acquisition system for early enamel erosion detection, the system comprising:
an image capturing device; and
a display device operatively connected to the image capturing device;
wherein the image acquisition system is configured to:
capture an image of a user's exposed teeth;
transmit the obtained image to a trained CNN that analyzes the obtained image by detecting and labeling dental pathologies to yield an analyzed image; and
receive and display the analyzed image on the display device.

10. The system of claim 9, further comprising a light source.

11. The system of claim 10, wherein the light source is configured to emit visible and near infrared light.

12. The system of claim 9, wherein the image capturing device is sensitive to visible and near infrared light sources.

13. The system of claim 9, wherein the image capture is based on a timer.

14. The system of claim 9, wherein the image capture is based on a voice command.

15. The system of claim 1, wherein the enamel erosion detection uses a pre-defined set of anchors specific to recognizing early enamel erosions at ratios 1:1, 1:1.4 and 1.4:1 in the scales of 24, 46 and 64 during region proposal.

16. A method for training an image recognition algorithm for early enamel erosion detection using the system of claim 1.
